(19) United States
(12) Patent Application Publication
(10) Pub. No.: US 2025/0049654 A1
(43) Pub. Date: Feb. 13, 2025

(54) COSMETIC COMPOSITION

(71) Applicant: SHISEIDO COMPANY, LTD., Tokyo (JP)

(72) Inventors: Satoshi YAMAKI, Yokohama (JP); Lethu NGUYEN, East Windsor, NJ (US); Alicja MIKULKO, East Windsor, NJ (US)

(73) Assignee: SHISEIDO COMPANY, LTD., Tokyo (JP)

(21) Appl. No.: 18/717,692

(22) PCT Filed: Dec. 2, 2022
(86) PCT No.: PCT/IB2022/061677

§ 371 Date: Jun. 7, 2024

Related U.S. Application Data
(60) Provisional application No. 63/287,820, filed on Dec. 9, 2021.

(51) Int. Cl. A61K 8/06; A61K 8/04; A61K 8/27; A61K 8/29; A61K 8/73; A61K 8/9717; A61Q 17/04

(52) U.S. Cl. CPC A61K 8/062; A61K 8/042; A61K 8/27; A61K 8/29; A61K 8/731; A61K 8/9717; A61Q 17/04; A61K 2800/30; A61K 2800/48

(57) ABSTRACT

Disclosed is a cosmetic composition that includes (a) a thickening system containing (a) at least one natural water gelling agent comprising a cellulose derivative; and (b) an oil. The composition does not contain a polyethylene glycol surfactant.

### CROSS-REFERENCE TO RELATED APPLICATIONS

The present application claims priority under 35 U.S.C. § 371 to International Patent Application No. PCT/IB2022/061677, filed on Dec. 2, 2022, which claims priority to U.S. Provisional Patent Application No. 63/287,820 filed Dec. 9, 2021. The contents of these applications are hereby incorporated by reference in their entireties.

### FIELD

The present disclosure relates in general to cosmetic compositions, and specifically to cosmetic compositions, which do not contain certain ingredients, such as a PEG surfactant, and their methods of making and methods of use.

### SUMMARY

One embodiment is a cosmetic composition comprising (a) a thickening system containing (a) at least one natural water gelling agent comprising a cellulose derivative; and (b) an oil, wherein the composition does not comprise a polyethylene glycol surfactant.

Another embodiment is a cosmetic composition (a) a thickening system containing (a) at least one natural water gelling agent comprising a cellulose derivative; and (b) an oil, wherein the composition does not comprise any ingredient selected from acrylates and acrylamide copolymers.

### FIGURES

FIG. 1 is a UV-Visible spectrum of a control composition that was unstable.

FIG. 2 is a UV-Visible spectrum of exemplary composition of Example 2.

### DETAILED DESCRIPTION

Unless otherwise specified “a” or “an” means one or more.

As used herein, the term “about” placed before a specific numeric value may mean±20% of the numeric value; ±18% of the numeric value, ±15% of the numeric value; ±12% of the numeric value; ±8% of the numeric value; ±5% of the numeric value; ±3% of the numeric value; ±2% of the numeric value; ±1% of the numeric value or ±0.5% of the numeric value.

As used herein, the expression “oil-in-water composition” and related expressions refers to an oil-in-water type emulsified composition in which an oil phase is dispersed in an aqueous (water) phase. As used herein, the expression “powder-in-oil-in-water composition” and related expressions refers to an oil-in-water type emulsified composition, in which a powder is dispersed in an oil phase, which is dispersed in an aqueous (water) phase.

As used herein, the expression “microplastic beads” refers to particles containing a solid polymer, to which additives or other substances may have been added, and where ≥1% w/w of particles have (i) all dimensions 0.1 μm≤x≤5 mm, or (ii), for fibres, a length of 0.3 μm≤x≤15 mm and length to diameter ratio of >3. The expression “particles containing a solid polymer” refers either (i) a particle of any composition with a continuous solid polymer surface coating of any thickness or (ii) particles of any composition with a solid polymer content of ≥1% w/w.

All content information for ingredients of compositions expressed as percent (%) refers to percent (%) by mass, relative to the total mass of the composition, unless specified otherwise.

New standards and regulations regarding safety and non-toxicity affected a number of ingredients used in cosmetic compositions. For example, recently many organic UV filters, such as Oxybenzone and Octonoxate, have been banned in a number of countries. Certain solvents, such as 1,4-dioxane and ethylene oxide, as well as impurity of polyethylene glycol (PEG) surfactants are also raising concerns. Polymeric ingredients, such as (meth)acrylates, i.e. methacrylates and acrylates, and (meth)acrylamide, i.e. methacrylamide and acrylamide, have also safety issues as well as microplastic environment issue. These new requirements for ingredients may make challenging to develop new cosmetic compositions, such as cosmetic compositions having watery texture and sun protection properties. For example, it may be challenging to develop cosmetic compositions having watery texture and/or sensation without using acrylates and acrylamide polymers. Obtaining emulsification of oil-in-water compositions or powder-in-oil-in-water compositions may be challenging as well.

The present disclosure provides a cosmetic composition which includes (a) a thickening system containing (a) at least one natural water gelling agent, including a cellulose derivative; and (b) an oil. The thickening system may further include at least one hydrophilic gelling agent, such as a polysaccharide, e.g. agar.

In many embodiments, the cosmetic composition may have watery texture. In many embodiments, the present composition may be free of (meth)acrylates (methacrylates and acrylates) or (meth)acrylamide polymers. In many embodiments, the present composition may contain no PEG based ingredients, such as PEG surfactant. In many embodiments, the present composition may be free of synthetic polymers. In many embodiments, the present composition may be free of any polyethylene glycol containing ingredients, such as PEG surfactants, any polymeric ingredients, such as (meth)acrylates or (meth)acrylamide; and/or any silicones, such as silicone solvents.

In some embodiments, the cosmetic composition may have sun protection properties. For example, the cosmetic composition may have a sun protection factor SPF at least 2 or at least 5 or at least 10 or at least 20 or at least 30 or at least 40 or at least 50 or at least 60 or at least 70 or at least 80 or at least 90 or at least 100.

The composition may be used in a number of color cosmetics products. For example, the present composition may be used in a skin care product, a sun screen product, a primer product; a foundation product, a hair product, a deodorant, a lip product, such as a lipstick or a lip balm. In some embodiments, the composition may be a concealer. In some embodiments, the composition may be a primer. In some embodiments, the composition may be a cream, such as a gel cream.

In some embodiments, the cosmetic composition may be used alone. In other words, the composition may be applied alone, without another composition, to a keratinous surface or substrate, such as skin, e.g. lips, eyelids, face, or hair, of a subject, such as a human.

In some embodiments, the composition, which may be a cosmetic composition, may be used together with another product, for example, a top coat, a primer, or a powder.

### Gelling Agent

The composition may include at least one hydrophilic gelling agent. Non-limiting examples of appropriate hydrophilic gelling agents include thermoreversible polysaccharides, e.g., agar, agarose, carrageenan, and gellan. The at least one hydrophilic gelling agent may be incorporated into the formulations in the powder form or gels.

In one exemplary embodiment, the at least one hydrophilic gelling agent include an agar, such as the one described in U.S. Pat. No. 8,933,134, which is hereby incorporated by reference. An agar, which may be used, may insoluble in cold water, but it swells considerably, absorbing as much as twenty times its own weight of water. It dissolves readily in boiling water and sets to a firm gel at concentrations as low as 0.50 percent. Another example may be powdered dry agar that is soluble in water and other solvents at temperatures between 95 degrees centigrade and 100 degrees centigrade And yet another example may be moistened agar flocculated by ethanol, 2-propanol or acetone, or salted out by high concentrations of electrolytes, that is soluble in a variety of solvents at room temperature. In some embodiments, agar may be in the form of a powder. In some embodiments, agar may be added to the composition as an agar gel. Preparation and characteristics of agar gels are disclosed in U.S. Pat. Nos. 9,757,312 and 8,367,044, all of which are hereby incorporated by reference in their entirety. The agar used may be of cosmetic as well as food grade, including those available from Ina Food Industry Corporation.

A content of the at least one hydrophilic gelling agent, such as agar, in the composition may vary. In some embodiments, an amount of the at least one hydrophilic gelling agent, such as agar, in the composition may be from 0.1 mass % to 5 mass % or from 0.1 mass % to 3 mass % or from 0.3 mass % to 3 mass % or from 0.5 mass % to 2 mass % or any range or subvalue within these ranges.

In some embodiments, the composition may be an oil-in-water type emulsified cosmetic composition, which contains an aqueous phase; and an oily phase dispersed in the aqueous phase.

In some embodiments, the composition may be a powder-in-oil-in water emulsion, which may include a powder, such as a hydrophobic powder dispersed in an oily phase, which in turn is dispersed in an aqueous phase. Powder-in-oil-in-water emulsions are disclosed for example, in WO2017057563.

The aqueous phase of such composition contains a polar solvent, such as water, and the thickening system comprising the at least one hydrophilic gelling agent, such as agar, and the cellulose derivative, while the oily phase of the composition contains the oil, such as a hydrocarbon oil, which may be, for example, an ester oil, a vegetable oil. The oily phase may also contain one or more organic UV absorbers.

A content of water in the composition may vary. In some embodiments, an amount of water in the composition may be from 5 mass % to 95 mass % or from 25 mass % to 75 mass % or from 30 mass % to 65 mass % or from 35 mass % to 55 mass % or any value or subrange within these ranges.

The cellulose derivative in the thickening system may provide stability and/or robustness the oil-in-water type emulsified cosmetic composition without a PEG surfactant.

In some embodiments, the cellulose derivative may be an amphiphilic cellulose derivative. In some embodiments, a cellulose derivative may be a hydroxypropyl-methylcellulose alkyl ether, such as a hydroxypropyl-methylcellulose C₁₅-C₂₀ alkyl ether, e.g. hydroxypropyl-methylcellulose stearoxy ether. Such cellulose derivative are disclosed, for example, in US 2012/0058061, which is incorporated herein by reference in its entirety.

In some embodiments, the cellulose derivative may have emulsifying properties. Such cellulose derivatives are disclosed, for example, in US 2020/0352845 and US 2020/0246230, each of which is incorporated by reference in its entirety.

For example, in some embodiments, the cellulose derivative may be hydrophobically Modified Alkylcellulose. The hydrophobically modified alkylcellulose may be one represented by the formula (II).

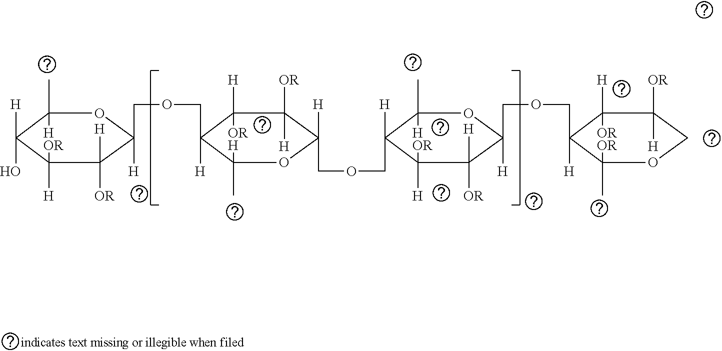

In the above formula (II), R is a bonded (composite) group R₁-R₂, R₁ is the same or different in the molecule, and is a group selected from —[CH₂CH(CH₃)O]ᵣ—, —[CH₂CH₂]ᵣ and —[CH₂CH(OH)CH₂]ᵣ— (wherein r is an integer of 0 to 4), R₂ is at least one group selected from a hydrocarbon group having 12 to 28 carbon atoms (preferably an alkyl group having 1 to 4 carbon atoms) and a hydrogen atom, and at least one R₂ in a molecule is a hydrocarbon group having 12 to 28 carbon atoms (preferably an alkyl group having 1 to 4 carbon atom). A is a group —(CH₂)ₜ— (wherein t is an integer of 1 to 3) and s is a number of 100 to 10,000.

The hydrophobically modified alkylcellulose of the formula (II) has a structure in which a long chain alkyl group, which is a hydrophobic group, is introduced into a water-soluble cellulose ether derivative through a polyoxyalkylene chain. Examples of water-soluble cellulose ether derivatives, which are the base of the molecule, include methylcellulose, ethyl cellulose, propyl cellulose, butyl cellulose, hydroxyethyl cellulose, Hydroxypropylcellulose and hydroxypropyl methylcellulose. The hydrophobically modified alkylcellulose may be prepared by reacting those with long chain alkyl glycidyl ether (e.g., those represented by the following formula (II′)).

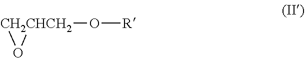

In the formula (II′), R′ is an alkyl group having 10 to 28, preferably 12 to 22 carbon atoms.

Hydroxypropyl methylcellulose or hydroxyethyl cellulose are preferred as the above water-soluble cellulose ether derivative. In particular, it is preferable to select hydroxypropyl methylcellulose. Furthermore, it is preferable that R′ in long chain alkyl glycidyl ether (II′) is a stearyl group (—C₁₈H₃₇) or a cetyl group (—C₁₆H₃₃) (in these cases, —CH₂CH(OH)CH₂OR′ is —CH₂CH(OH)CH₂O—C₁₈H₃₇ or —CH₂CH(OH)CH₂O—C₁₆H₃₃).

One example of the hydrophobically modified alkylcellulose (component B-2) may be stearoxy hydroxypropyl methylcellulose (INCI name: Hydroxypropylmethylcellulose Stearoxy Ether) in which the hydrophobic group R′ in the formula (II′) is a stearyl group. A product with the product name “SANGELOSE” commercially available from Daido Chemical Corporation may also be used. (Product names: SANGELOSE 90L, 90M, 90H, 60L, 60M, 60H and the like).

In some embodiments, the cellulose derivative may enhance the emulsifying system and contribute to the stability of finished products, including those that are characterized as oil-in-water emulsified compositions (0/W) and powder-in-oil-in-water emulsified compositions (P/0/W) For example, in some embodiments adding 0.04% of Sangelose 90L may decrease a failure ratio of emulsification of a powder-in-oil-in-water emulsified composition from 50% to 0%.

An amount of the cellulose derivative in the composition may vary. In some embodiments, an amount of the cellulose derivative, such as hydroxypropyl-methylcellulose alkyl ether, in the composition may be from 0.01 mass % to 4 mass % or from 0.01 mass % to 2 mass % or from 0.01 mass % to 1 mass % or from 0.02 mass % to 1 mass % or from 0.05 mass % to 0.5 mass % or any value or subrange within these ranges. For example, in some embodiments, an amount of hydroxypropyl-methylcellulose C₁₅-C₂₀ alkyl ether, such as hydroxypropyl-methylcellulose stearoxy ether, in the composition may be from 0.01 mass % to 4 mass % or from 0.01 mass % to 2 mass % or from 0.01 mass % to 1 mass % or from 0.02 mass % to 1 mass % or from 0.05 mass % to 0.5 mass % or any value or subrange within these ranges.

In some embodiments, a mass ratio between the at least one hydrophilic gelling agent, such as agar, and the cellulose derivative, such as hydroxypropyl-methylcellulose alkyl ether, in the composition may be from 100:1 to 0.5:1 or from 90:1 to 0.5:0.9 or from 80:1 to 0.5:0.8 or from 50:1 to 1:1 or from 30:1 to 2:1 or any value or subrange within these ranges. For example, a mass ratio between the at least one hydrophilic gelling agent, such as agar, and hydroxypropyl-methylcellulose C₁₅-C₂₀ alkyl ether, such as hydroxypropyl-methylcellulose stearoxy ether, in the composition may be from 100:1 to 0.5:1 or from 90:1 to 0.5:0.9 or from 80:1 to 0.5:0.8 or from 50:1 to 1:1 or from 30:1 to 2:1 or 20:1 to 5:1 or any value or subrange within these ranges.

A combination of the cellulose derivative, such as hydroxypropyl methylcellulose stearoxy ether, and the at least one hydrophilic gelling agent, such as agar, may provide the composition with watery texture and/or sensation. Such combination may also reduce water leaking which may occur in agar-containing compositions.

In some embodiments, the thickening system may include cellulose gum.

### Oil

In some embodiments, the at least one oil may comprise at least hydrocarbon oil.

A content of the at least oil, such as the at least one hydrocarbon oil, in the composition may vary. In some embodiments, an amount of the at least one oil, such as the at least one hydrocarbon oil, in the composition may be from 5 mass % to 50 mass % or from 8 mass % to 50 mass % or from 10 mass % to 40 mass 5 or from 12 mass % to 30 mass % or from 15 mass % to 26 mass % or any value or subrange within these ranges.

In some embodiments, the oil may comprise a volatile oil and a non-volatile oil. In some embodiments, the oil may comprise a volatile hydrocarbon oil and a non-volatile hydrocarbon oil.

A volatile oil, such a volatile hydrocarbon oil, refers to an oil, such as hydrocarbon oil, having a boiling point lower than 300° C. at 1 atm. Examples of volatile hydrocarbon oils include, but not limited to, C₉-C₁₄ alkanes, such as isoparaffins, isododecane and isohexadecane. In some embodiments, a volatile hydrocarbon oil may be Vegelight 1214LC, which is C₉₋₁₂ Alkanes (and) Coco-caprylate/caprate.

A non-volatile oil, such as non-volatile hydrocarbon oil, refers to an oil, such as a hydrocarbon oil, having a boiling point of 300° C. or higher at 1 atm, Examples of non-volatile hydrocarbon oils include, but not limited to, light squalane, hydrogenated polydecene and vaseline.

A content of a volatile oil, such as a volatile hydrocarbon oil, in the composition may vary. For example, in some embodiments, an amount of a volatile oil, such as a volatile hydrocarbon oil may be from 3 mass % to 6 mass % or from 6 mass % to 30 mass % or from 5 mass to 25 mass % or from 8 mass % to 25 mass % or from 8 mass % to 20 mass % or from 10 mass % to 20 mass % or any value or subrange within these ranges.

A content of a non-volatile oil, such as a non-volatile hydrocarbon oil, in the composition may vary. For example, in some embodiments, an amount of a non-volatile oil, such a non-volatile hydrocarbon oil, may be from 2 mass to 40 mass % or from 3 mass % to 30 mass % or from 4 mass % to 20 mass % or from 2 mass % to 20 mass % or from 3 mass % to 15 mass % or from 4 mass % to 10 mass % or any value or subrange within these ranges.

A mass ratio between a volatile oil, such as a volatile hydrocarbon oil, and a non-volatile oil, such as a non-volatile hydrocarbon oil, may vary. In some embodiments, a mass ratio between a volatile oil, such as a volatile hydrocarbon oil, and a non-volatile oil, such as a non-volatile hydrocarbon oil, may be from 1:10 to 10:1 or from 1:5 to 10:1 or from 1:5 to 5:1 or from 1:2 to 5:1 or from 1:1 to 3:1 or any value or subrange within these ranges. In some embodiments, the composition may contain only a non-volatile oil and no volatile oil. Yet in some embodiments, the composition may contain only a volatile oil and no non-volatile oil.

### Surfactant

In some embodiments, the composition may comprise at least one non-PEG based surfactant, such as at least one polyglyceryl surfactant. In some embodiments, the composition may contain no surfactant for emulsification. In such case, a cellulose derivative, such as Sangelose, may provide emulsification.

The at least one polyglyceryl surfactant may be dissolved or dispersed in water of the aqueous phase. A combination of the at least one non-PEG based surfactant, such as at least one polyglyceryl surfactant, together with the cellulose derivative, such as hydroxypropyl methylcellulose stearoxy ether, may provide the composition emulsification stability as an oil-in-water composition or a powder-in oil-in-water composition without using a PEG surfactant.

At least one polyglyceryl surfactant may be selected, for example, from polyglyceryl 2 surfactants, polyglyceryl 3 surfactants, polyglyceryl 4 surfactants, polyglyceryl-5 surfactants, polyglyceryl-6 surfactants, polyglyceryl-7 surfactants, polyglyceryl-8 surfactants, polyglyceryl 9 surfactants, polyglyceryl 10 surfactants, polyglyceryl 11 surfactants, and polyglyceryl 12 surfactants.

Exemplary polyglyceryl surfactants include, but not limited to, Polyglyceryl-4 Caprate, Polyglyceryl-2 Caprate, Polyglyceryl-4 Caprylate, Polyglyceryl-6 Caprylate, Polyglyceryl-6 Caprate, Polyglyceryl-4 Caprylate/Caprate, Polyglyceryl-6 Caprylate/Caprate, Polyglyceryl-3 Cocoate, Polyglyceryl-4 Cocoate, Polyglyceryl-10 Decalinoleate, Polyglyceryl-10 Decaoleate, Polyglyceryl-10 Decacasterate, Polyglyceryl-3 Dicaprate, Polyglyceryl-3 Dicocoate, Polyglyceryl-10 Didecanoate, Polyglyceryl-2 Diisostearate, Polyglyceryl-3 Diisostearate, Polyglyceryl-10 Diisostearate, Polyglyceryl-4 Dilaurate, Polyglycerin-2 Dioleate, Polyglyceryl-3 Dioleate, Polyglyceryl-6 Dioleate, Polyglyceryl-10 Dioleate, Polyglyceryl-6 Dipalmitate, Polyglyceryl-10 Dipalmitate, Polyglyceryl-2 Dipolyhydroxystearate, Polyglyceryl-2 Distearate, Polyglyceryl-3 Distearate, Polyglyceryl-6 Distearate, Polyglyceryl-10 Distearate, Polyglyceryl-10 Heptaoleate, Polyglyceryl-10 Heptastearate, Polyglyceryl-6 Hexaoleate, Polyglyceryl-10 Hexaoleate, Polyglyceryl-2 Isopalmitate, Polyglyceryl-2 Isostearate, Polyglyceryl-4 Isostearate, Polyglyceryl-5 Isostearate, Polyglyceryl-6 Isostearate, Polyglyceryl-10 Isostearate, Polyglyceryl-2 Laurate, Polyglyceryl-3 Laurate, Polyglyceryl-4 Laurate, Polyglyceryl-4 Laurate/Sebacate, Polyglyceryl-4 Laurate/Succinate, Polyglyceryl-5 Laurate, Polyglyceryl-6 Laurate, Polyglyceryl-10 Laurate, Polyglyceryl-3 Myristate, Polyglyceryl-10 Myristate, Polyglyceryl-2 Oleate, Polyglyceryl-3 Oleate, Polyglyceryl-4 Oleate, Polyglyceryl-5 Oleate, Polyglyceryl-6 Oleate, Polyglyceryl-8 Oleate, Polyglyceryl-10 Oleate, Polyglyceryl-3 Palmitate, Polyglyceryl-6 Palmitate, Polyglyceryl-10 Pentalaurate, Polyglyceryl-10 Pentalinoleate, Polyglyceryl-4 Pentaoleate, Polyglyceryl-10 Pentaoleate, Polyglyceryl-3 Pentaricinoleate, Polyglyceryl-6 Pentaricinoleate, Polyglyceryl-10 Pentaricinoleate, Polyglyceryl-4 Pentastearate, Polyglyceryl-6 Pentastearate, Polyglyceryl-10 Pentastearate, Polyglyceryl-3 Polyrisinoleate, Polyglyceryl-6 Polyricinoleate, Polyglyceryl-3 Ricinoleate, Polyglyceryl-2 Sesquiisostearate, Polyglyceryl-2 Sesquioleate, Polyglyceryl-2 Sesquistearate, Polyglyceryl-3 Stearate, Polyglyceryl-2 Stearate, Polyglyceryl-4 Stearate, Polyglyceryl-8 Stearate, Polyglyceryl-10 Stearate, Polyglyceryl-2 Tetraisostearate, Polyglyceryl-6 Tetraoleate, Polyglyceryl-10 Tetraoleate, Polyglyceryl-2 Tetrastearate, Polyglyceryl-2 Triisostearate, Polyglyceryl-3 Triisostearate, Polyglyceryl-10 Trioleate, Polyglyceryl-4 Tristearate, Polyglyceryl Tri stearate, and Polyglyceryl-10 Tristearate.

In some embodiments, at least one polyglyceryl surfactants may include at least one polyglyceryl 4 surfactant, such as polyglyceryl-4 laurate/succinate.

An amount of the at least one polyglyceryl surfactant in the composition may vary. In some embodiments, the at least one polyglyceryl surfactant may constitute from 0 mass % to 6 mass % or from 0.3 mass % to 6 mass % or from 0.1 mass % to 4 mass % or 0.3 mass % to 3 mass % or from 0.5 mass % to 4 mass % or from 1 mass % to 3 mass % or any value or subrange within these ranges of the composition.

### Powders

In some embodiments, the composition may include one or more powders. For example, when the composition is an oil-in-water type emulsified cosmetic composition, which contains an aqueous phase containing the thickening system; and an oily phase, which contains the oil and is dispersed in the aqueous phase, the one or more powders may be dispersed in the oily phase. The one or more powders may include one or more surface treated powders, such as one or more hydrophobically surface treated powders. Hydrophobic treatment of outer surfaces of particles in the powder may facilitate dispersion of the powder in the oily phase.

In some embodiments, one or more powders may include one or more of titanium dioxide particles, silica particle, iron oxide particles and zinc oxide particles. A hydrophobization surface treatment applied to one or more powders, such as titanium dioxide particles, silica particles, iron oxide particles and/or zinc oxide particles, may be (1) a treatment with a metallic soap consisting of a higher fatty acid and a multivalent metal, such as a divalent, e.g. magnesium, or a trivalent metal, e.g. aluminum or (2) a composite treatment with a higher fatty acid and a hydroxide of a multivalent metal, such as a divalent metal, e.g. magnesium or a trivalent metal, e.g. aluminum. The higher fatty acid may be a C8 to C24, such as C12 to C22, linear or branched carboxylic acid, e.g. stearic acid or isostearic acid. For example, in some embodiments, one or more powders, such as titanium dioxide particles, silica particles iron oxide particles and/or zinc oxide particles, may be surface treated with magnesium stearate and/or magnesium isostearate or with a composite treatment of aluminum hydroxide and stearic and/or isostearic acid.

In some embodiments, one or more powders may provide at least some of sun protection properties, such as ultraviolet protection properties, to the composition. For examples, the one or more powders may comprise one or more ultraviolet scattering powders. Examples of ultraviolet scattering powders include titanium dioxide powders, zinc oxide powders, silica powders, and composite powders, such as titanium dioxide coated mica, titanium dioxide coated bismuth oxychloride, titanium dioxide coated talc and titanium dioxide coated glass flake. Ultraviolet scattering powders may have an average particle diameter of about 25 to 100 nm. In some embodiments, one or more ultraviolet scattering powders may include trititanium dioxide powder, zinc oxide powder or a combination thereof.

Ultraviolet scattering powders may hydrophobic treated on a surface of a base material, such as zinc oxide or titanium dioxide. Examples of methods of hydrophobic treatment of surface include silicone treatment using methylhydrogen polysiloxane, methyl polysiloxane, trimethylsiloxysilicic acid, silicone resin or the like; fluorine treatment using perfluoroalkyl phosphoric acid ester, perfluoroalcohol or the like; amino acid treatment using N-acylglutamic acid or the like; lecithin treatment; metallic soap treatment; fatty acid treatment; and alkylphosphoric acid ester treatment.

A content of ultraviolet scattering powder(s) in the composition may vary. In some embodiments, an amount of the ultraviolet scattering powder(s) may be from 2.5 mass % to 30 mass % or from 3 mass % to 25% or from 4 mass % to 20% mass % or any value or subrange within these ranges.

### Organic UV Absorbers

In some embodiments, the composition may comprise one or more organic ultraviolet absorbers. In such case, the one or more organic ultraviolet absorbers may provide at least some of sun protection properties, such as ultraviolet protecting properties, to the composition.

In some embodiments, one or more organic ultraviolet absorbers may include oil-soluble and oil-dispersible organic ultraviolet absorbers, which may be dissolved and/or dispersed in the oil phase of the composition.

In some embodiments, one or more organic ultraviolet absorbers may include water soluble and/or water dispersible organic ultraviolet absorbers, which may be dissolved and/or dispersed in the aqueous phase. One non-limiting example of such absorbers is phenylbenzimidazole sulfonic acid Itrade name: EUSOLEX 232, Merck KGaA).

Examples of oil-soluble and/or oil-dispersible ultraviolet absorbers include, but not limited to benzoic acid derivatives, salicylic acid derivatives, cinnamic acid derivatives, dibenzoylmethane derivatives, β-β-diphenyl acrylate derivatives, benzophenone derivatives, benzylidene camphor derivatives, phenylbenzimidazole derivatives, triazine derivatives, phenylbenzotriazole derivatives, anthranil derivatives, imidazoline derivatives, benzal malonate derivatives, and 4,4-diarylbutadiene derivatives.

Examples of benzoic acid derivatives include, but not limited to ethyl p-aminobenzoate (PABA), ethyl dihydroxypropyl PABA, ethylhexyl dimethyl PABA (e.g., “Escalol™. 507; ISP), glyceryl PABA, PEG-25 PABA (e.g., “Uvinul™ P25”; BASF), and diethylamino hydroxybenzoyl hexyl benzoate (e.g., “Uvinul™ A Plus”).

Examples of salicylic acid derivatives include, but not limited to homosalate (“Eusolex™ HMS”; Rona/EM Industries, Inc.), ethylhexyl salicylate (e.g., “Neo Heliopan™ OS”; Haarmann & Reimer), dipropylene glycol salicylate (e.g., “Dipsal™”; Scher), and TEA-salicylate (e.g., “Neo Heliopan™ TS”; Haarmann & Reimer).

Examples of cinnamic acid derivatives include, but not limited to octyl methoxycinnamate or ethylhexyl methoxycinnamate (e.g., “Parsol™ MCX”; Hoffmann-La Roche, Ltd.), isopropyl methoxycinnamate, isoamyl methoxycinnamate (e.g., “Neo Heliopan™E1000”; Haarmann & Reimer), cinoxate, DEA methoxycinnamate, diisopropyl methylcinnamate, glyceryl ethylhexanoate dimethoxycinnamate, and di-(2-ethylhexyl)-4′-methoxybenzal malonate.

Examples of dibenzoylmethane derivatives include, but not limited to 4-tert-butyl-4′-methoxydibenzoylmethane (e.g., “Parsol™ 1789”).

Examples of β-β-diphenyl acrylate derivatives include octocrylene (e.g., Uvinul™ N539”; BASF).

Examples of benzophenone derivatives include benzophenone-1 (e.g., “Uvinul™ 400”; BASF), benzophenone-2 (e.g., “Uvinul™ D50”; BASF), benzophenone-3 or oxybenzone (e.g., “Uvinul™ M40”; BASF), benzophenone-4 (e.g., “Uvinul™ MS40”; BASF), benzophenone-5, benzophenone-6 (e.g., “Helisorb™ 11”; Norquay Technology Inc.), benzophenone-8 (e.g., “Spectra-Sorb™ UV-24”; American Cyanamid Co.), benzophenone-9 (e.g., “Uvinul™DS-49”; BASF), and benzophenone-12.

Examples of the benzylidene camphor derivatives include 3-benzylidene camphor (e.g., “Mexoryl™ SD”; Chimex), 4-methylbenzylidene camphor, benzylidene camphor sulfonic acid (e.g., “Mexoryl™ SL”; Chimex), camphor benzalkonium methosulfate (e.g., “Mexoryl™ SO”; Chimex), terephthalylidene dicamphor sulfonic acid (e.g., “Mexoryl™ SX”; Chimex), and polyacrylamide methylbenzylidene camphor (e.g., “Mexoryl™ SW”; Chimex).

Examples of phenylbenzimidazole derivatives include phenylbenzimidazole sulfonic acid (e.g., “Eusolex™ 232”; Merck KGaA), and disodium phenyl dibenzimidazole tetrasulfonate (e.g., “Neo Heliopan™ AP”; Haarmann & Reimer).

Examples of triazine derivatives include anisotriazine (e.g., “Tinosorb™ 5”; Ciba Specialty Chemicals Inc.), ethylhexyl triazone (e.g., “Uvinul™ T-150”; BASF), diethylhexyl butamido triazone (e.g., “Uvasorb™ HEB”; 3V SIGMA S.p.A.), and 2,4,6-tris(diisobutyl-4′-aminobenzalmalonate)-s-triazine.

Examples of phenylbenzotriazole derivatives include drometrizole trisiloxane (e.g., “Silatrizole™ Rhodia Chimie), and methylene bis-benzotriazolyl tetramethylbutylphenol (e.g., “Tinosorb™ M”; Ciba Specialty Chemicals Inc.).

Examples of anthanil derivatives include menthyl anthranilate (e.g., “Neo Heliopan™ MA”; Haarmann & Reimer).

Examples of imidazoline derivatives include ethyihexyl dimethoxybenzylidene dioxoimidazoline propionate.

Examples of the benzal malonate derivatives include polyorganosiloxane having a benzal malonate functional group (e.g., Polysilicone-15; “Parsol™ SLX”; DSM Nutrition Japan K.K.).

Examples of the 4,4-diarylbutadiene derivatives include 1,1-dicarboxy(2,2′-dimethylpropyl)-4,4-diphenylbutadiene.

A content of the one or more organic ultraviolet absorbers, such as one or more oil soluble and/or oil-dispersible ultraviolet absorbers, may vary. In some embodiments, the composition may contain no such absorbers at all. In such case, sun protection properties of the composition may be due to ultraviolet scattering powder(s). Yet in some embodiments, the composition may contain the one or more organic ultraviolet absorbers, such as one or more oil soluble and/or oil-dispersible ultraviolet absorbers, in an amount from 1 mass % to 30 mass % or from 1 mass % to 25 mass % or from 3 mass % to 25 mass % or from 3 mass % to 20 mass % or from 5 mass to 23 mass 5 or from 5 mass % to 18 mass %.

### Pigments

In some embodiments, the composition may also include one or more pigments. In some embodiments, one or more pigments may be in a form of a powder. In some embodiments, a powder used as a pigment may be a hydrophobically surface treated powder, which may be dispersed in the oily phase of the composition. Yet in some embodiments, a powder used as a pigment may be a hydrophilically surface treated powder, which may be dispersed in the aqueous phase of the composition.

In some embodiments, a powder used as a pigment may be a pigment grade powder. The term “pigment grade” may mean an average particle size in the powder of about 0.2 to 0.4 μm. In some embodiments, one or more pigments may be selected from pigment-grade titanium dioxide, pigment-grade zinc oxide, fine particle zinc oxide, talc, mica, sericite, kaolin, titanated mica, black iron oxide, yellow iron oxide, red iron oxide, ultramarine, Prussian blue, chromium oxide, chromium hydroxide, silica and cerium oxide. In some embodiments, one or more pigments may include one or more mineral pigments, such as one or more iron oxide pigments, such as black iron oxide, yellow iron oxide and red iron oxide.

In some embodiments, one or more mineral pigments, such iron oxide pigments, may be hydrophobically surface treated with, for example, lauroyl lysine. Lauroyl lysine treated iron oxide pigments are commercially available, for example, as Unipure Yellow LC 182 LL, Unipure Red LC 381 LL and Unipure Black LC 989 LL.

A content of the one or more pigments in the composition may vary. For example, in some embodiments, an amount of one or more pigments in the composition may be from 0.1 mass % to 20 mass % or from 0.2 mass % to 15 mass % or from 0.5 mass % to 10 mass % or from 0.1 mass % to 5 mass % or from be from 0.2 mass % to 4 mass % or from 0.3 mas % to 3 mass % or from 0.5 mass % to 2 mass % or any value or subrange within these ranges.

### Excluded Ingredients

In some embodiments, the composition may be free of microplastic particles. The expression “microplastic particles” may mean particles containing solid polymer, to which additives or other substances may have been added, and where ≥1% w/w of particles have (i) all dimensions 0.1 μm≤x≤5 mm, or (ii), for fibres, a length of 0.3 μm≤x≤15 mm and length to diameter ratio of >3. The expression “particles containing solid polymer” may mean either (i) a particle of any composition with a continuous solid polymer surface coating of any thickness or (ii) particles of any composition with a solid polymer content of ≥1% w/w.”

In some embodiments, the composition may be free of (meth)acrylate and/or (meth)acrylamide copolymer. Such composition may still have watery feeling and/or sensation.

In some embodiments, the composition may be free of any PEG-based ingredient, such as a PEG-based surfactant. As an oil-in-water type emulsified composition or as a powder-in-oil-in-water emulsified composition, the composition without a PEG-based surfactant may still have emulsification stability.

In some embodiments, the composition may be free of silicone, such as silicone solvent.

### EXEMPLARY COMPOSITION

In some embodiments, the composition may be an oil-in-water type emulsified cosmetic composition comprising: an aqueous phase; an oily phase dispersed in the aqueous phase, and a hydrophobic powder dispersed in the oily phase, wherein the aqueous phase contains water and the thickening system and the oily phase contains the oil, wherein the oil comprises a volatile hydrophobic oil and a non-volatile hydrophobic oil, wherein the cellulose derivative is hydroxypropyl methylcellulose stearoxy ether and wherein a mass ratio between agar and hydroxypropyl methylcellulose stearoxy ether is the composition is from 50:1 to 1:1.

### Additional Ingredients

In some embodiments, the composition may include one or more additional ingredients, such as a filler, which may be silica particles; a preservative, such as phenoxyethanol; a preservative boosting surfactant, such as propanediol; a natural polymer, such as a natural polysaccharide, e.g. succinoglycan; a humectant, an emollient and/or a moisturizer, such as glycerin.

### Method of Making

The composition may be prepared by mixing water and the thickening system comprising at least one hydrophilic gelling agent, such as agar, and the cellulose derivative; mixing one or more oil of the oil phase, such as a volatile oil, e.g. a volatile hydrocarbon oil, and/or a non-volatile oil, e.g. a non-volatile hydrocarbon oil. Optionally, additional ingredients may be added and/or dispersed in the oil phase. Such additional ingredients may include, for example, powders, including ultraviolet scattering powders, and pigments. The oil phase may be added to the water phase and the addition product may be emulsified using for example, a homogenizer.

Embodiments described herein are further illustrated by, though in no way limited to, the following working examples.

### Example 1

Two compositions presented in Table 1 were prepared. A Manufacturing Process involved (a) mixing a water phase and a water thickener (thickening system) (b) mixing an oil phase and dispersing powders, pigments in the oil phase; (c) adding the oil phase to the water phase and emulsifying the product by a homogenizer

TABLE 1

| | | Ex. Comp. | Ex. Comp |
|---|---|---|---|
| Material name | Substance Identification | 1 | 2 |
| DEIONIZED WATER | WATER | To 100 | To 100 |
| INA AGAR CS-110 | AGAR | 1 | 1 |
| NATRAGEM E145 NP- | POLYGLYCERYL-4 | 1 | 2 |
| LQ-(MV) | LAURATE/SUCCINATE & | | |
| | WATER | | |
| ZEMEA | PROPANEDIOL | 8 | 8 |
| PHENOXETOL NF 272556 | PHENOXYETHANOL | 0.7 | 0.7 |
| SANGELOSE 90L | HYDROXYPROPYL | 0.1 | 0.1 |
| | METHYLCELLULOSE | | |
| | STEAROXY ETHER | | |
| DEIONIZED WATER | WATER | 3 | 6 |
| NEOSSANCE | SQUALANE | 6 | 9 |
| SQUALANE | | | |
| VEGELIGHT 1214LC | COCONUT ALKANES & COCO- | 13 | 18 |
| | CAPRYLATE/CAPRATE | | |
| ESTEMOL 182V | SORBITAN | 1 | 1 |
| | SESQUIISOSTEARATE | | |
| ISOSTEARIC ACID SX | ISOSTEARIC ACID | 1 | 1 |
| TITANIUM DIOXIDE MT- | Titanium Dioxide & Aluminum | 7.5 | 10 |
| 100 TV | Hydroxide & | | |
| | Stearic Acid | | |
| TAYCA MP-25 MG | TITANIUM DIOXIDE & | 4 | 4 |
| | MAGNESIUM STEARATE | | |
| UNIPURE YELLOW LC | IRON OXIDES (duplicate 2) & | 1 | 1 |
| 182 LL | LAUROYL LYSINE | | |
| UNIPURE RED LC 381 LL | IRON OXIDES (duplicate 1) & | 0.5 | 0.5 |
| | LAUROYL LYSINE | | |
| UNIPURE BLACK LC 989 | IRON OXIDES (duplicate 3) & | 0.1 | 0.1 |
| LL | LAUROYL LYSINE | | |
| SATINIER M5 | SILICA | 1 | 1 |
| AQUALON ™ CMC 7M2F | CELLULOSE GUM | 0.3 | 0.3 |
| (ASHLAND SPECIALTY | | | |
| INGREDIENTS) | | | |
| RHEOZAN | SUCCINOGLYCAN | 0.1 | 0.1 |

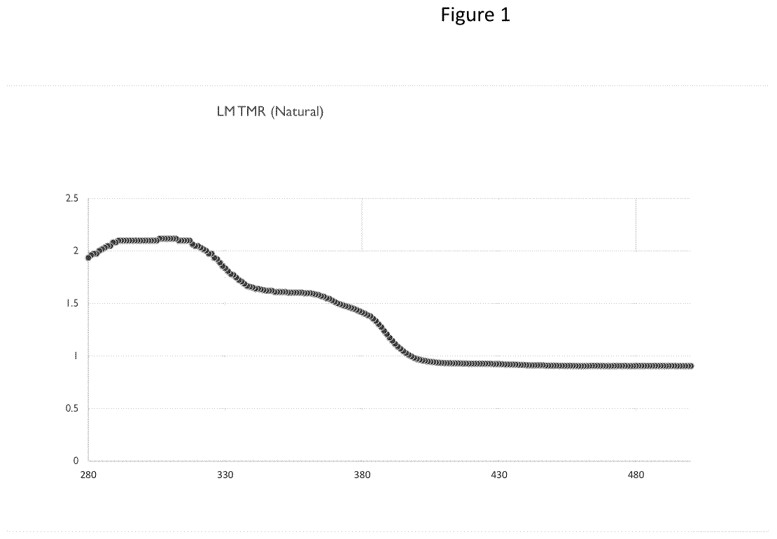

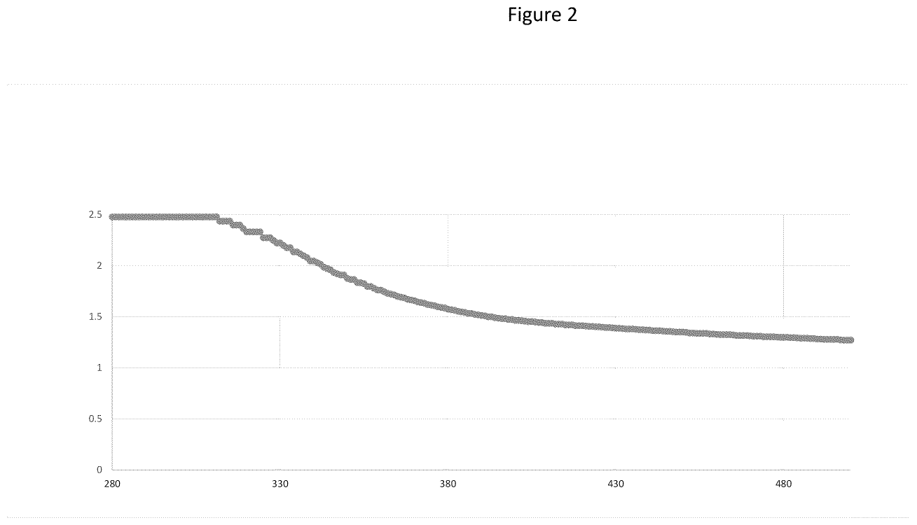

FIG. 2 provide UV-visible spectrum of Exemplary Composition 2 measured by HITACH U-3900H Spectrophotometer. Based on the data in FIGS. 1 and 2, the compositions in Table 1 have ultraviolet protection properties.

The combination of a cellulose derivative, such as hydroxypropyl methylcellulose stearoxy ether, and agar allows obtaining watery texture without acrylates and/or acrylamide polymers. Also these compositions can be applied a watery SPF tinted moisturizer with skincare sensory.

### Example 2

A number of compositions without acrylates and acrylamide polymers has been manufactured. The compositions are summarized in Table 2. A Manufacturing Process involved mixing a water phase and water thickener (thickening system); mixing an oil phase; adding the oil phase to the water phase and emulsifying the added product by a homogenizer at 6000 rpm.

The compositions of Table 2 were evaluated for a number of properties, including watery texture and sensation.

Evaluation:

1. Emulsification:
+: Average emulsion size is less than 5 μm,
+/−: Average emulsion size is more than 5 μm,
−: Emulsification is failed
2. Watery sensation while application
+: More than 70% subjects felt the watery sensation
+/−: More than 40% subjects felt the watery sensation
−: Less than 40% subjects felt the watery sensation
3. Natural origin polymers
Yes: Contained only natural origin polymers
No: Contained synthetic polymers
4. Water leaking at 50 C. 4 weeks
+: No water leaking on the top
+/−: Tiny water leaking on the top
−: Noticeable water leaking on the top

TABLE 2
Watery texture and sensation without acrylates and acrylamide polymers

| | Substance | Control | Control | Control | Control | Control | Control | Example |
|---|---|---|---|---|---|---|---|---|
| Trade name | Identification | Comp 1 | Comp. 2 | Comp 3 | Comp 4 | Comp 5 | Comp 6 | Comp 1 |
| DEIONIZED WATER | WATER | To 100 | To 100 | To 100 | To 100 | To 100 | To 100 | To 100 |
| GLYCERINE NAT 99.7% | GLYCERIN | 5 | 5 | 5 | 5 | 5 | 5 | 5 |
| ACIDCHEM | | | | | | | | |
| ZEMEA | PROPANEDIOL | 4 | 4 | 4 | 4 | 4 | 4 | 4 |
| HCO-100FF | Peg-100 | 1 | | | 1 | | | |
| | Hydrogenated | | | | | | | |
| | Castor Oil | | | | | | | |
| NIKKOL TS-10V | Polysorbate 60 | | 1 | | | | | |
| NATRAGEM E145 NP-LQ- | POLYGLYCERYL-4 | | | | | | | 1 |
| (MV) | LAURATE/ | | | | | | | |
| | SUCCINATE | | | | | | | |
| ESSERAN 200 | DIISOPROPYL | 15 | 15 | 15 | 15 | 15 | 15 | 15 |
| | SEBACATE | | | | | | | |
| NEOSSANCE SQUALANE | SQUALANE | 15 | 15 | 15 | 15 | 15 | 15 | 15 |
| CARBOPOL 980 | Carbomer | 0.5 | 0.5 | 0.5 | | | | |
| POLYMER | | | | | | | | |
| PEMULEN ™ TR-2 | Acrylates/C10-30 | | | 0.2 | | | | |
| POLYMER | Alkyl Acrylate | | | | | | | |
| | Crosspolymer | | | | | | | |
| SU POLYMER G-1 | Dimethylacrylamide/ | | | | 0.5 | | | |
| | Sodium | | | | | | | |
| | Acryloyldimethyl- | | | | | | | |
| | taurate | | | | | | | |
| | Crosspolymer | | | | | | | |
| ARISTOFLEX AVC | Ammonium | | | | | 0.5 | | |
| | Acryloyldimethyl- | | | | | | | |
| | taurate/VP | | | | | | | |
| | Copolymer | | | | | | | |
| ARISTOFLEX HMB | Ammonium | | | | | | 0.5 | |
| | Acryloyldimethyl- | | | | | | | |
| | taurate/Beheneth- | | | | | | | |
| | 25 Methacrylate | | | | | | | |
| | Crosspolymer | | | | | | | |
| INA AGAR CS-110 | AGAR | | | | | | | 1 |
| AQUALON ™ CMC 7M2F | CELLULOSE | | | | | | | |
| | GUM | | | | | | | |
| RHEOZAN | SUCCINOGLYCAN | | | | | | | |
| SANGELOSE 90L | HYDROXYPROPYL | | | | | | | |
| | METHYLCELLULOSE | | | | | | | |
| | STEAROXY ETHER | | | | | | | |
| CAUSTIC POTASH DRY | POTASSIUM | 0.05 | 0.05 | 0.05 | | | | |
| | HYDROXIDE | | | | | | | |
| Evaluation | Emulsification | + | + | +/− | + | +/− | +/− | +/− |
| | Watery sensation while applying | + | + | + | + | + | + | + |
| | Natural origin polymers | No | No | No | No | No | No | Yes |
| | Stability (Water leaking at 50 C. | + | + | + | + | + | + | − |
| | 4 weeks) | | | | | | | |

| | Substance | Example | Control | Example | Example | Example |
|---|---|---|---|---|---|---|
| Trade name | Identification | Comp 2 | Comp 7 | Comp 3 | Comp 4 | Comp 5 |
| DEIONIZED WATER | WATER | To 100 | To 100 | To 100 | To 100 | To 100 |
| GLYCERINE NAT 99.7% | GLYCERIN | 5 | 5 | 5 | 5 | 5 |
| ACIDCHEM | | | | | | |
| ZEMEA | PROPANEDIOL | 4 | 4 | 4 | 4 | 4 |
| HCO-100FF | Peg-100 | | | | | |
| | Hydrogenated | | | | | |
| | Castor Oil | | | | | |
| NIKKOL TS-10V | Polysorbate 60 | | | | | |
| NATRAGEM E145 NP-LQ- | POLYGLYCERYL-4 | 1 | 1 | | 1 | |
| (MV) | LAURATE/ | | | | | |
| | SUCCINATE | | | | | |
| ESSERAN 200 | DIISOPROPYL | 15 | 15 | 15 | 15 | 15 |
| | SEBACATE | | | | | |
| NEOSSANCE SQUALANE | SQUALANE | 15 | 15 | 15 | 15 | 15 |
| CARBOPOL 980 | Carbomer | | | | | |
| POLYMER | | | | | | |
| PEMULEN ™ TR-2 | Acrylates/C10-30 | | | | | |
| POLYMER | Alkyl Acrylate | | | | | |
| | Crosspolymer | | | | | |
| SU POLYMER G-1 | Dimethylacrylamide/ | | | | | |
| | Sodium | | | | | |
| | Acryloyldimethyl- | | | | | |
| | taurate | | | | | |
| | Crosspolymer | | | | | |
| ARISTOFLEX AVC | Ammonium | | | | | |
| | Acryloyldimethyl- | | | | | |
| | taurate/VP | | | | | |
| | Copolymer | | | | | |
| ARISTOFLEX HMB | Ammonium | | | | | |
| | Acryloyldimethyl- | | | | | |
| | taurate/Beheneth- | | | | | |
| | 25 Methacrylate | | | | | |
| | Crosspolymer | | | | | |
| INA AGAR CS-110 | AGAR | | | | 1 | 1 |
| AQUALON ™ CMC 7M2F | CELLULOSE | 1 | | | 0.5 | |
| | GUM | | | | | |
| RHEOZAN | SUCCINOGLYCAN | | 1 | | | |
| SANGELOSE 90L | HYDROXYPROPYL | | | 1 | | 0.5 |
| | METHYLCELLULOSE | | | | | |
| | STEAROXY ETHER | | | | | |
| CAUSTIC POTASH DRY | POTASSIUM | | | | | |
| | HYDROXIDE | | | | | |
| Evaluation | Emulsification | +/− | +/− | +/− | + | + |
| | Watery sensation while applying | +/− | − | +/− | + | + |
| | Natural origin polymers | Yes | Yes | Yes | Yes | Yes |
| | Stability (Water leaking at 50 C. | + | + | + | +/− | +/− |
| | 4 weeks) | | | | | |

Result: Non-PEG emulsification and combination of Agar and Cellulose derivatives showed good sensory, stability and emulsification capability with high natural property.

### Example 3

A number of powder-in-oil-in-water emulsion compositions without PEG surfactant has been manufactured. The compositions are summarized in Table 3. A Manufacturing Process involved (a) mixing a water phase and a water thickener (thickening system) (b) mixing an oil phase and dispersing powders, pigments, inorganic UV filters in the oil phase; (c) adding the oil phase to the water phase and emulsifying the product by a homogenizer.

The compositions were evaluated for a number of properties, including emulsification instability and water leakage prevention.

Evaluation:

Emulsification:
+: Average emulsion size is less than 5 μm,
+/−: Average emulsion size is more than 5 μm,
−: Emulsification is failed
2. Viscosity: Measured by Blookfield DV-1 Viscometer (LV-04, 12 prm)
3. Watery sensation while application
+: at least 70% subjects felt the watery sensation
+/−: at least 40% but less than 70% of subjects felt the watery sensation
−: Less than 40% subjects felt the watery sensation
4. Natural origin polymers
Yes: Contained only natural origin polymers
No: Contained synthetic polymers
5. Stability (Water leaking at 50C 4 weeks)
+: No water leaking on the top
+/−: Tiny water leaking on the top
−: Noticeable water leaking on the top
6. UV protective effect (Absorbance at 320 nm): Applied 25 mg samples
on PMMA plate and measured the absorbance by HITACH U-3900H
Spectrophotometer

TABLE 3
Emulsification instability for P/O/W system without PEG surfactant and prevention of water leaking by Agar gel

| | Substance | Control | Control | Control | Ex. | Ex. |
|---|---|---|---|---|---|---|
| Trade name | Identification | Comp 8 | Comp 9 | Comp 10 | Comp 6 | Comp 7 |
| DEIONIZED WATER | WATER | To 100 | To 100 | To 100 | To 100 | To 100 |
| GLYCERINE NAT | GLYCERIN | 2 | 2 | 2 | 2 | 2 |
| 99.7% ACIDCHEM | | | | | | |
| PHENOXETOL NF | PHENOXYETHANOL | 0.5 | 0.5 | 0.5 | 0.5 | 0.5 |
| 272556 | | | | | | |
| NATRAGEM E145 NP- | POLYGLYCERYL-4 | | | | 1 | 1 |
| LQ-(MV) | LAURATE/ | | | | | |
| | SUCCINATE | | | | | |
| | & WATER | | | | | |
| ZEMEA | PROPANEDIOL | 10 | 10 | 10 | 10 | 10 |
| SU POLYMER G-1 | Dimethylacrylamide/ | 0.4 | | | | |
| | Sodium | | | | | |
| | Acryloyldimethyl- | | | | | |
| | taurate Crosspolymer | | | | | |
| SANGELOSE 90L | HYDROXYPROPYL | 0.2 | 0.2 | 0.3 | 0.05 | 0.2 |
| | METHYLCELLULOSE | | | | | |
| | STEAROXY ETHER | | | | | |
| AQUALON ™ CMC | CELLULOSE | | | 1 | 0.2 | 0.2 |
| 7M2F | GUM | | | | | |
| RHEOZAN | SUCCINOGLYCAN | | 0.2 | | 0.1 | 0.1 |
| INA AGAR CS-110 | AGAR | | | | 1.1 | 1.1 |
| NEOSSANCE | SQUALANE | 5 | 5 | 5 | 5 | 5 |
| SQUALANE | | | | | | |
| VEGELIGHT C912-LC | C9-12 ALKANE, | 12 | 12 | 12 | 12 | 12 |
| (GRANT INDUSTRIES) | COCO- | | | | | |
| | CAPRYLATE/CAPRATE | | | | | |
| ESTEMOL 182V | SORBITAN | 0.5 | 0.5 | 0.5 | 0.5 | 0.5 |
| | SESQUIISOSTEARATE | | | | | |
| ISOSTEARIC ACID SX | ISOSTEARIC | 1 | 1 | 1 | 1 | 1 |
| | ACID | | | | | |
| TITANIUM DIOXIDE | Titanium Dioxide & | 7.5 | 7.5 | 7.5 | 7.5 | 7.5 |
| MT-100 TV | Aluminum | | | | | |
| | Hydroxide & | | | | | |
| | Stearic Acid | | | | | |
| TAYCA MP-25 MG | TITANIUM | 3 | 3 | 3 | 3 | 3 |
| | DIOXIDE & | | | | | |
| | MAGNESIUM | | | | | |
| | STEARATE | | | | | |
| UNIPURE YELLOW LC | IRON OXIDES | 1 | 1 | 1 | 1 | 1 |
| 182 LL | (duplicate 2) & | | | | | |
| | LAUROYL | | | | | |
| | LYSINE | | | | | |
| UNIPURE RED LC 381 | IRON OXIDES | 0.5 | 0.5 | 0.5 | 0.5 | 0.5 |
| LL | (duplicate 1) & | | | | | |
| | LAUROYL | | | | | |
| | LYSINE | | | | | |
| UNIPURE BLACK LC | IRON OXIDES | 0.1 | 0.1 | 0.1 | 0.1 | 0.1 |
| 989 LL | (duplicate 3) & | | | | | |
| | LAUROYL | | | | | |
| | LYSINE | | | | | |
| SATINIER M5 | SILICA | 2 | 2 | 2 | 2 | 2 |
| Evaluation | Emulsification | +/− | +/− | +/− | + | + |
| | Viscosity | 16000 | 12150 | 21000 | 28700 | 41750 |
| | Watery sensation while applying | + | +/− | +/− | +/− | + |
| | Natural origin polymers | No | Yes | Yes | Yes | Yes |
| | Stability (Water leaking at 50 C. | + | + | + | +/− | +/− |
| | 4 weeks) | | | | | |
| | UV protective effect (Absorbance | 0.92 | 0.88 | 0.91 | 2.01 | 2.02 |
| | at 320 nm) | | | | | |
| | Substance | Ex. | Ex. | Ex. | Ex. | Ex. |
| Trade name | Identification | Comp 8 | Comp 9 | Comp 10 | Comp 11 | Comp 12 |
| DEIONIZED WATER | WATER | To 100 | To 100 | To 100 | To 100 | To 100 |
| GLYCERINE NAT | GLYCERIN | 2 | 2 | 2 | 2 | 2 |
| 99.7% ACIDCHEM | | | | | | |
| PHENOXETOL NF | PHENOXYETHANOL | 0.5 | 0.5 | 0.5 | 0.5 | 0.5 |
| 272556 | | | | | | |
| NATRAGEM E145 NP- | POLYGLYCERYL-4 | 1 | 1.5 | 1.5 | 1.5 | 1 |
| LQ-(MV) | LAURATE/ | | | | | |
| | SUCCINATE | | | | | |
| | & WATER | | | | | |
| ZEMEA | PROPANEDIOL | 10 | 10 | 10 | 10 | 10 |
| SU POLYMER G-1 | Dimethylacrylamide/ | | | | | |
| | Sodium | | | | | |
| | Acryloyldimethyl- | | | | | |
| | taurate Crosspolymer | | | | | |
| SANGELOSE 90L | HYDROXYPROPYL | 0.1 | 0.1 | 0.1 | 0.1 | 0.2 |
| | METHYLCELLULOSE | | | | | |
| | STEAROXY ETHER | | | | | |
| AQUALON ™ CMC | CELLULOSE | 0.2 | 0.2 | 0.2 | 0.2 | 0.2 |
| 7M2F | GUM | | | | | |
| RHEOZAN | SUCCINOGLYCAN | 0.1 | 0.1 | 0.1 | 0.1 | 0.1 |
| INA AGAR CS-110 | AGAR | 1.1 | 1.1 | 0.8 | 0.6 | 0.6 |
| NEOSSANCE | SQUALANE | 5 | 8 | 8 | 8 | 5 |
| SQUALANE | | | | | | |
| VEGELIGHT C912-LC | C9-12 ALKANE, | 12 | 15 | 15 | 15 | 12 |
| (GRANT INDUSTRIES) | COCO- | | | | | |
| | CAPRYLATE/CAPRATE | | | | | |
| ESTEMOL 182V | SORBITAN | 0.5 | 0.5 | 0.5 | 0.5 | 0.5 |
| | SESQUIISOSTEARATE | | | | | |
| ISOSTEARIC ACID SX | ISOSTEARIC | 1 | 1 | 1 | 1 | 1 |
| | ACID | | | | | |
| TITANIUM DIOXIDE | Titanium Dioxide & | 7.5 | 10 | 10 | 10 | 7.5 |
| MT-100 TV | Aluminum | | | | | |
| | Hydroxide & | | | | | |
| | Stearic Acid | | | | | |
| TAYCA MP-25 MG | TITANIUM | 3 | 3 | 3 | 3 | 3 |
| | DIOXIDE & | | | | | |
| | MAGNESIUM | | | | | |
| | STEARATE | | | | | |
| UNIPURE YELLOW LC | IRON OXIDES | 1 | 1 | 1 | 1 | 1 |
| 182 LL | (duplicate 2) & | | | | | |
| | LAUROYL | | | | | |
| | LYSINE | | | | | |
| UNIPURE RED LC 381 | IRON OXIDES | 0.5 | 0.5 | 0.5 | 0.5 | 0.5 |
| LL | (duplicate 1) & | | | | | |
| | LAUROYL | | | | | |
| | LYSINE | | | | | |
| UNIPURE BLACK LC | IRON OXIDES | 0.1 | 0.1 | 0.1 | 0.1 | 0.1 |
| 989 LL | (duplicate 3) & | | | | | |
| | LAUROYL | | | | | |
| | LYSINE | | | | | |
| SATINIER M5 | SILICA | 2 | 2 | 2 | 2 | 2 |
| Evaluation | Emulsification | + | + | + | + | + |
| | Viscosity | 33350 | 28300 | 28300 | 28300 | 28300 |
| | Watery sensation while applying | + | + | + | + | + |
| | Natural origin polymers | Yes | Yes | Yes | Yes | Yes |
| | Stability (Water leaking at 50 C. | +/− | +/− | + | + | + |
| | 4 weeks) | | | | | |
| | UV protective effect (Absorbance | 2.03 | 2.48 | 1.4 | 1.67 | 1.08 |
| | at 320 nm) | | | | | |

Results: The combination of Agar and HYDROXYPROPYL METHYLCELLULOSE STEAROXY ETHER achieved high viscosity and good watery sensation. Combination of non-PEG surfactant and HYDROXYPROPYL METHYLCELLULOSE STEAROXY ETHER showed good emulsification capability of P/O/W system. Combination of Agar, HYDROXYPROPYL METHYLCELLULOSE STEAROXY ETHER, inorganic UV filters showed strong UV protection with SPF boosting effect. Increasing the ratio of agar to Hydroxypropyl methylcellulose stearoxy ether resulted in better stability.

### Example 4

A number of additional compositions has been manufactured. The compositions are presented in Table 4. Manufacturing Process was as follows (a) Mix a water phase and a water thickener (thickening system); (b) mix an oil phase and disperse powders, pigments, inorganic UV filters in the oil phase; (c) add the oil phase to water phase and emulsify the product by a homogenizer.

A number of properties has been evaluated for the manufactured compositions.

Evaluation:

Emulsification:
+: Average emulsion size is less than 5 μm,
+/−: Average emulsion size is more than 5 μm,
−: Emulsification is failed
2. Viscosity: Measured by Blookfield DV-1 Viscometer (LV-04, 12 prm)
3. Watery sensation while application
+: More than 70% subjects felt the watery sensation
+/−: More than 40% subjects felt the watery sensation
−: Less than 40% subjects felt the watery sensation
4. Natural origin polymers
Yes: Contained only natural origin polymers
No: Contained synthetic polymers
5. Stability (Water leaking at 50 C. 4 weeks)
+: No water leaking on the top
+/−: Tiny water leaking on the top
−: Noticeable water leaking on the top
6. UV protective effect (Absobance at 320 nm): Applied 25 mg samples
on PMMA plate and measured the absorbance by HITACH U-3900H
Spectrophotometer

TABLE 4
Variations of Compositions

| | | Ex. | Ex. | Ex. | Ex. | Ex. |
|---|---|---|---|---|---|---|
| Trade name | Substance Identification | Comp 13 | Comp14 | Comp 15 | Comp 16 | Comp 17 |
| DEIONIZED WATER | WATER | To 100 | To 100 | To 100 | To 100 | To 100 |
| INA AGAR CS-110 | AGAR | 0.6 | 0.6 | 0.6 | 0.6 | 0.6 |
| GLYCERINE NAT 99.7% ACIDCHEM | GLYCERIN | 3 | 3 | 3 | 3 | 3 |
| NATRAGEM E145 NP-LQ-(MV) | POLYGLYCERYL-4 | 1.5 | 1.5 | 1.5 | 1.5 | 1.5 |
| | LAURATE/SUCCINATE & WATER | | | | | |
| ZEMEA | PROPANEDIOL | 8 | 8 | 8 | 8 | 8 |
| PHENOXETOL NF 272556 | PHENOXYETHANOL | 0.4 | 0.4 | 0.4 | 0.4 | 0.4 |
| SANGELOSE 90L | HYDROXYPROPYL | 0.1 | 0.1 | 0.1 | 0.1 | 0.1 |
| | METHYLCELLULOSE STEAROXY | | | | | |
| | ETHER | | | | | |
| NEOSSANCE SQUALANE | SQUALANE | 8 | 8 | 8 | 8 | 8 |
| NEO HELIOPAN HMS HOMSLTE | HOMOSALATE | | 1.4 | | | 1.4 |
| NEO HELIOPAN OS OCTISLTE | ETHYLHEXYL SALICYLATE | 5 | 5 | 5 | 5 | 5 |
| PARSOL 340 | Octocrylene | 5 | 5 | 5 | 5 | 5 |
| VEGELIGHT C912-LC (GRANT | C9-12 ALKANE, COCO- | 5 | 5 | 5 | 5 | 5 |
| INDUSTRIES) | CAPRYLATE/CAPRATE | | | | | |
| ESTEMOL 182V | SORBITAN SESQUIISOSTEARATE | 0.5 | 0.5 | 0.5 | 0.5 | 0.5 |
| ISOSTEARIC ACID SX | ISOSTEARIC ACID | 1 | 1 | 1 | 1 | 1 |
| TITANIUM DIOXIDE MT-100 TV | Titanium Dioxide & Aluminum Hydroxide & | 5 | 3 | 5 | 8 | 5 |
| | Stearic Acid | | | | | |
| ASL-1 TIO2 CR-50 | Ci 77491 & Sodium Dilauramidoglutamide | 3 | 3 | | | |
| | Lysine & Magnesium Chloride | | | | | |
| ASL-1 YELLOW LL-100P | Ci 77492 & Sodium Dilauramidoglutamide | 0.7 | 0.7 | | | |
| | Lysine & Magnesium Chloride | | | | | |
| ASL-1 RED R-516P | Ci 77491 & Sodium Dilauramidoglutamide | 0.3 | 0.3 | | | |
| | Lysine & Magnesium Chloride | | | | | |
| ASL-1 BLACK BL-100P | Ci 77499 & Sodium Dilauramidoglutamide | 0.1 | 0.1 | | | |
| | Lysine & Magnesium Chloride | | | | | |
| SATINIER M5 | SILICA | 2 | 2 | 2 | 2 | 2 |
| AQUALONTM CMC 7M2F | CELLULOSE GUM | 0.3 | 0.3 | 0.3 | 0.3 | 0.3 |
| RHEOZAN | SUCCINOGLYCAN | 0.1 | 0.1 | 0.1 | 0.1 | 0.1 |
| Evaluation | Emulsification | + | + | + | + | + |
| | Viscosity | | 18050 | 17900 | 18050 | 19200 |
| | Watery sensation while applying | | + | + | + | + |
| | Natural origin polymers | | No | Yes | Yes | Yes |
| | Stability (Water leaking at 50 C. 4 weeks) | | +/− | +/− | + | + |
| | UV protective effect (Absobance at 320 nm) | | 1.25 | 1.15 | 1.21 | 1.62 |

Result: This system can be applied to organic UV filters and non-color SPF formula as well.

### Example 5

A number of additional compositions has been manufactured, see tables 5-7 using the manufacturing process similar to those of Examples 2-4.

TABLE 5
Concealer (P/O/W)

| | | Ex. Comp | |
|---|---|---|---|
| Trade name | Substance Identification | 18 | Ex. Comp 19 |
| DEIONIZED WATER | WATER | To 100 | To 101 |
| INA AGAR CS-110 | AGAR | 0.6 | 0.6 |
| GLYCERINE NAT 99.7% ACIDCHEM | GLYCERIN | 4 | 4 |
| NATRAGEM E145 NP-LQ-(MV) | POLYGLYCERYL-4 LAURATE/SUCCINATE & WATER | 2 | 2 |
| DIPROPYLENE GLYCOL | DIPROPYLENE GLYCOL | 10 | 10 |
| FRAGRANFCE GRADE | | | |
| PHENOXETOL NF 272556 | PHENOXYETHANOL | 0.4 | 0.4 |
| SANGELOSE 90L | HYDROXYPROPYL METHYLCELLULOSE STEAROXY ETHER | 0.1 | 0.1 |
| NEOSSANCE SQUALANE | SQUALANE | 10 | 10 |
| VEGELIGHT 1214LC | COCONUT ALKANES & COCO-CAPRYLATE/CAPRATE | 15 | 15 |
| ESTEMOL 182V | SORBITAN SESQUIISOSTEARATE | 0.5 | 0.5 |
| ISOSTEARIC ACID SX | ISOSTEARIC ACID | 1 | 1 |
| ASL-1 TIO2 CR-50 | Ci 77491 & Sodium Dilauramidoglutamide | 8 | 12 |
| | Lysine & Magnesium Chloride | | |
| ASL-1 YELLOW LL-100P | Ci 77492 & Sodium Dilauramidoglutamide | 3 | 5 |
| | Lysine & Magnesium Chloride | | |
| ASL-1 RED R-516P | Ci 77491 & Sodium Dilauramidoglutamide | 2 | 3 |
| | Lysine & Magnesium Chloride | | |
| ASL-1 BLACK BL-100P | Ci 77499 & Sodium Dilauramidoglutamide | 0.5 | 0.6 |
| | Lysine & Magnesium Chloride | | |
| SATINIER M5 | SILICA | 2 | 2 |
| AQUALON ™ CMC 7M2F | CELLULOSE GUM | 0.3 | 0.3 |
| RHEOZAN | SUCCINOGLYCAN | 0.3 | 0.3 |

TABLE 6
High transparent primer (O/W)

| Trade name | Substance Identification | Ex. Comp 20 | Ex. Comp 21 |
|---|---|---|---|
| DEIONIZED WATER | WATER | To 100 | To 100 |
| INA AGAR CS-110 | AGAR | 1.5 | 2 |
| GLYCERINE NAT 99.7% ACIDCHEM | GLYCERIN | 3 | 3 |
| NATRAGEM E145 NP-LQ-(MV) | POLYGLYCERYL-4 LAURATE/SUCCINATE & WATER | 1 | 1 |
| ZEMEA | PROPANEDIOL | 8 | 8 |
| PHENOXETOL NF 272556 | PHENOXYETHANOL | 0.4 | 0.4 |
| SANGELOSE 60L | HYDROXYPROPYL METHYLCELLULOSE | 0.2 | 0.3 |
| | STEAROXY ETHER | | |
| NEOSSANCE SQUALANE | SQUALANE | 8 | 8 |
| NEO HELIOPAN HMS HOMSLTE | HOMOSALATE | | 10 |
| NEO HELIOPAN OS OCTISLTE | ETHYLHEXYL SALICYLATE | 5 | 5 |
| PARSOL 340 | Octocrylene | 5 | 5 |
| PARSOL ® 1789 | BUTYL METHOXYDIBENZOYLMETHANE/AVOBENZONE | 2 | 2.5 |
| VEGELIGHT 1214LC | COCONUT ALKANES & COCO-CAPRYLATE/CAPRATE | 5 | 5 |
| ESTEMOL 182V | SORBITAN SESQUIISOSTEARATE | 0.5 | 0.5 |
| ISOSTEARIC ACID SX | ISOSTEARIC ACID | 1 | 1 |
| TITANIUM DIOXIDE MT-100 TV | Titanium Dioxide & Aluminum Hydroxide & Stearic Acid | 5 | 8 |
| SOLESPHERE L-51S | SILICA | 2 | 2 |
| RHEOZAN | SUCCINOGLYCAN | 0.5 | 0.5 |

TABLE 7
Skincare gel cream (O/W)

| | | Ex Comp | Ex. Comp |
|---|---|---|---|
| Trade name | Substance Identification | 22 | 23 |
| DEIONIZED WATER | WATER | To 100 | To 100 |
| INA AGAR CS-110 | AGAR | 2 | 2.5 |
| GLYCERINE NAT 99.7% ACIDCHEM | GLYCERIN | 10 | 10 |
| NATRAGEM E145 NP-LQ-(MV) | POLYGLYCERYL-4 LAURATE/SUCCINATE & WATER | 2 | 2 |
| 1,3 BUTYLENE GLYCOL | BUTYLENE GLYCOL | 8 | 8 |
| PHENOXETOL NF 272556 | PHENOXYETHANOL | 0.4 | 0.4 |
| AQUALON ™ CMC 7M2F | CELLULOSE GUM | 0.2 | 0.3 |
| ESSERAN 200 | DIISOPROPYL SEBACATE | 8 | 8 |
| PRIPURE 3759-LQ-(GD) | SQUALANE | 10 | 10 |
| CRODAMOL OSU-LQ-(JP) | DIETHYLHEXYL SUCCINATE | 5 | 5 |
| VEGELIGHT C912-LC (GRANT INDUSTRIES) | C9-12 ALKANE, COCO-CAPRYLATE/CAPRATE | 5 | 5 |
| TITANIUM DIOXIDE MT-100 TV | Titanium Dioxide & Aluminum Hydroxide & Stearic Acid | 5 | 8 |

### Example 6

TABLE 8

| | | Ex. | Ex. | Ex. | Ex. | Ex. |
|---|---|---|---|---|---|---|
| TRADE NAME | INCI | 22 | 23 | 24 | 25 | 26 |
| DEIONIZED | WATER | To | To | To | To | To |
| WATER | | 100 | 100 | 100 | 100 | 100 |
| INA AGAR CS-110 | AGAR | 0.8 | 0.8 | 0.8 | 0.8 | 0.8 |
| GLYCERINE | GLYCERIN | 1.5 | 1.5 | 1.5 | 1.5 | 1.5 |
| NAT 99.7% | | | | | | |
| ACIDCHEM | | | | | | |
| 1,3 | BUTYELENE | 8 | 8 | 8 | | |
| BUTYELENE | GLYCOL | | | | | |
| GLYCOL | | | | | | |
| OLIVEM 1000 | CETEARYL OLIVATE, | | | | 2 | |
| MB | SORBITAN OLIVATE | | | | | |
| BATYL | BATYL ALCOHOL | | | | 2 | |
| ALCOHOL EX | | | | | | |
| TEGOSOFT PC | POLYGLYCERYL 4- | | | | | 1.5 |
| 41MB | CAPRATE | | | | | |
| ZEMEA | PROPANEDIOL | 5 | 5 | 5 | 5 | 5 |
| PHENOXETOL | PHENOXYETHANOL | 0.4 | 0.4 | 0.4 | 0.4 | 0.4 |
| NF 272556 | | | | | | |
| SANGELOSE | HYDROXYPROPYL | 0.1 | 0.15 | 0.2 | 0.1 | 0.1 |
| 90L | METHYLCELLULOSE | | | | | |
| | STEAROXY ETHER | | | | | |
| NEOSSANCE | SQUALANE | 10 | 10 | 10 | 10 | 10 |
| SQUALANE | | | | | | |
| VEGELIGHT | C9-12 ALKANE, | 15 | 15 | 15 | 15 | 15 |
| C912-LC | COCO- | | | | | |
| (GRANT | CAPRYLATE/ | | | | | |
| INDUSTRIES) | CAPRATE | | | | | |
| ESTEMOL | SORBITAN | 0.5 | 0.5 | 0.5 | 0.5 | 0.5 |
| 182V | SESQUIISOSTEARATE | | | | | |
| ISOSTEARIC | ISOSTEARIC ACID | 1 | 1 | 1 | 1 | 1 |
| ACID SX | | | | | | |
| TITANIUM | Titanium Dioxide & | 10 | 10 | 10 | 10 | 10 |
| DIOXIDE MT- | Aluminum Hydroxide & | | | | | |
| 100 TV | Stearic Acid | | | | | |
| TAYCA MP-25 | TITANIUM | 3 | 3 | 3 | 3 | 3 |
| MG | DIOXIDE & | | | | | |
| | MAGNESIUM | | | | | |
| | STEARATE | | | | | |
| UNIPURE | IRON OXIDES | 1 | 1 | 1 | 1 | 1 |
| YELLOW LC | (duplicate 2) & | | | | | |
| 182 LL | LAUROYL LYSINE | | | | | |
| UNIPURE RED | IRON OXIDES | 0.5 | 0.5 | 0.5 | 0.5 | 0.5 |
| LC 381 LL | (duplicate 1) & | | | | | |
| (PULVERIZED) | LAUROYL LYSINE | | | | | |
| UNIPURE | IRON OXIDES | 0.1 | 0.1 | 0.1 | 0.1 | 0.1 |
| BLACK LC 989 | (duplicate 3) & | | | | | |
| LL (PULVERIZED) | LAUROYL LYSINE | | | | | |
| SATINIER M5 | SILICA | 1 | 1 | 1 | 1 | 1 |
| ZEMEA | PROPANEDIOL | 4 | 4 | 4 | 4 | 4 |
| AQUALON ™ | CELLULOSE GUM | 0.2 | 0.2 | 0.2 | 0.2 | 0.2 |
| CMC 7M2F | | | | | | |
| (ASHLAND | | | | | | |
| SPECIALTY | | | | | | |
| INGREDIENTS) | | | | | | |
| RHEOZAN | SUCCINOGLYCAN | 0.1 | 0.1 | 0.1 | 0.1 | 0.1 |

| | | | | | |
|---|---|---|---|---|---|
| Emulsification | +/− | +/− | +/− | + | + |
| Viscosity, mPa · s | 19000 | 25000 | 29000 | 30500 | 23400 |
| Watery sensation while applying | + | + | + | +/− | + |
| Natural origin polymers | Yes | Yes | Yes | Yes | Yes |
| Stability (Water leaking at 50 C. 4 weeks) | + | + | + | + | + |
| UV protective effect (Absobance at 320 nm) | 1.52 | 1.5 | 1.49 | 1.62 | 1.56 |

The compositions of Table 8 were prepared using the following Manufacturing Process: 1. Mix the water phase and water thickener; 2. Mix oil phase and disperse powders, G-3C, pigments, inorganic UV filters in the oil phase. 3. Add oil phase to water phase Emulsify by a homogenizer.

The compositions of Table 8 were evaluated as follows:

Evaluation:

1. Emulsification:
+: Average emulsion size is less than 5 μm,
+/−: +: average emulsion size is more than 5 μm,
−: Emulsification is failed
2. Viscosity: Measured by Blookfield DV-1 Viscometer (LV-04, 12 prm)
3. Watery sensation while application
+: At least 70% subjects felt the watery sensation
+/−: At least than 40% but less than 70% of subjects felt the watery
sensation
−: Less than 40% subjects felt the watery sensation
4. Natural origin polymers
Yes: Contained only natural origin polymers
No: Contained synthatic polymers
5. Stability (Water leaking at 50 C. 4 weeks)
+: No water leaking on the top
+/−: Tiny water leaking on the top
−: Noticeable water leaking on the top
6. UV protective effect (Absobance at 320 nm): Applied 25 mg samples
on PMMA plate and measured the absorbance by HITACH U-3900H
Spectrophotometer

The Results of Table 8 show that: the HYDROXYPROPYL METHYLCELLULOSE STEAROXY ETHER can emulsify P/0/W system. Other non-PEG emulsification system, OLIVEM and TEGOSOFT, showed good emulsification property.

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention.

All of the publications, patent applications and patents cited in this specification are incorporated herein by reference in their entirety.

1. A cosmetic composition comprising (a) a thickening system containing (a) at least one natural water gelling agent comprising a cellulose derivative; and (b) an oil, wherein the composition does not comprise a polyethylene glycol surfactant.

2. A cosmetic composition (a) a thickening system containing (a) at least one natural water gelling agent comprising a cellulose derivative; and (b) an oil, wherein the composition does not comprise any ingredient selected from acrylates and acrylamide copolymers.

3. The cosmetic composition of claim 1, which is an oil-in-water type emulsified cosmetic composition comprising: an aqueous phase; and an oily phase dispersed in the aqueous phase, wherein the aqueous phase contains the thickening system and the oily phase contains the oil.

4. The cosmetic composition of claim 3, further comprising a powder dispersed in the oily phase.

5. The cosmetic composition of claim 4, wherein the powder comprises hydrophobically treated powder.

6. The cosmetic composition of claim 5, wherein the hydrophobically treated powder comprises hydrophobically treated titanium oxide particles, hydrophobically treated zinc oxide or a combination thereof.

7. The cosmetic composition of claim 1, wherein the cellulose derivative comprises an amphiphilic cellulose derivative.

8. The cosmetic composition of claim 1, wherein the cellulose derivative comprises hydroxypropyl methylcellulose stearoxy ether.

9. The cosmetic composition of claim 1, wherein the oil comprises a hydrocarbon oil.

10. The cosmetic composition of claim 1, wherein the oil comprises a volatile oil and a non-volatile oil.

11. The cosmetic composition of claim 1, further comprising at least one polyglyceryl surfactant.

12. The cosmetic composition of claim 1, further comprising one or more pigments.

13. The cosmetic composition of claim 1, which is free of microplastic particles.

14. The cosmetic composition of claim 1, which is a sunscreen composition.

15. The cosmetic composition of claim 1, wherein the thickening system further comprises one hydrophilic gelling agent.

16. The cosmetic composition of claim 15, wherein the at least one hydrophilic gelling agent comprises agar.

17. The cosmetic composition of claim 15, wherein a content of the at least one hydrophilic gelling agent in the composition is from 0.1 mass % to 3 mass % and wherein a content of the cellulose derivative in the composition is from 0.01 mass % to 1 mass %.

18. The cosmetic composition of claim 16, wherein the cellulose derivative is hydroxypropyl methylcellulose stearoxy ether and wherein a mass ratio between agar and hydroxypropyl methylcellulose stearoxy ether is the composition is from 50:1 to 1:1.

19. The cosmetic composition of claim 1, which is an oil-in-water type emulsified cosmetic composition comprising: an aqueous phase; an oily phase dispersed in the aqueous phase, and a hydrophobic powder dispersed in the oily phase, wherein the aqueous phase contains the thickening system and the oily phase contains the oil, wherein the oil comprises a volatile hydrophobic oil and a non-volatile hydrophobic oil, wherein the cellulose derivative is hydroxypropyl methylcellulose stearoxy ether and wherein a mass ratio between the at least one hydrophilic gelling agent and hydroxypropyl methylcellulose stearoxy ether is the composition is from 50:1 to 1:1.

20. The cosmetic composition of claim 1 further comprising one or more organic ultraviolet absorbers.

21. The cosmetic composition of claim 20, wherein an amount of the one or more organic ultraviolet absorbers from 1 mass % to 25 mass %.

22. The cosmetic composition of claim 1, comprising
0.05 mass % to 0.5 mass % of hydroxypropyl methylcellulose stearoxy ether;
0.5 mass % to 2 mass % of agar;
10 mass % to 20 mass % of a volatile hydrocarbon oil;
4 mass % to 10 mass % of a non-volatile hydrocarbon oil; and
1 mass % to 3 mass % of at least one polyglyceryl surfactant.

23. A cosmetic method comprising applying the cosmetic composition of claim 1 to a keratinous surface.
